(19) United States
(12) Patent Application Publication
(10) Pub. No.: US 2021/0232914 A1
(43) Pub. Date: Jul. 29, 2021

(54) METHOD FOR BUILDING A HEART RHYTHM CLASSIFICATION MODEL

(71) Applicant: Ever Fortune.AI CO., Ltd., Taichung City (TW)

(72) Inventors: Kuan-Cheng CHANG, Taichung City (TW); Tzung-Chi HUANG, Taichung City (TW); Ken Ying-Kai LIAO, Taichung City (TW); Shih-Tsung HO, Taichung City (TW)

(21) Appl. No.: 17/145,000

(22) Filed: Jan. 8, 2021

Related U.S. Application Data
(60) Provisional application No. 62/962,802, filed on Jan. 17, 2020.

(51) Int. Cl. G06N 3/08; G06N 3/04; G16H 50/20; G16H 10/60; G16H 50/70; G16H 50/30; A61B 5/346

(52) U.S. Cl. CPC G06N 3/08; G06N 3/0445; G16H 50/20; A61B 5/346; G16H 50/70; G16H 50/30; G16H 10/60

(57) ABSTRACT

A method for building a heart rhythm classification model that is used to classify a heart rhythm of a person is provided. 12-lead ECG datasets are used to train a neural network model that includes multiple bidirectional LSTM layers. The bidirectional LSTM layers enable the neural network model to analyze the 12-lead ECG datasets in different aspects, so as to enhance classification accuracy.

### CROSS-REFERENCE TO RELATED APPLICATION

This application claims priority of U.S. Provisional Patent Application No. 62/962,802, filed on Jan. 17, 2020.

### FIELD

The disclosure relates to interpretation of a medical dataset, and more particularly to using a neural network model to interpret a 12-lead ECG dataset.

### BACKGROUND

Machine-learning technology has been widely used in image interpretation, speech recognition, item matching, and the presentation of relevant results during a search. Deep learning, which is one type of machine learning, involves an artificial neural network (ANN) with multilayer representation learning. In automatic interpretation of cardiovascular images, deep learning has been developed to interpret the results of electrocardiography (ECG), echocardiography, coronary computed tomography, and single-photon emission computed tomography for the evaluation of myocardial perfusion.

Electrocardiography is a graph of voltage versus time of the electrical activity of the heart using electrodes placed on the skin, and is widely used to detect various heart diseases, including rhythm disorders, conduction abnormalities, and myocardial ischemia or infarction.

In an article by Mathews, S. M., Kambhamettu, C. and Barner, K. E., “A novel application of deep learning for single-lead ECG classification,” Comput Biol Med. 99, 53-62 (2018), a machine-learning based method was proposed to detect and classify different types of cardiac arrhythmias using a single-lead ECG dataset. In an article by Hannun, A. Y. et al., “Cardiologist-level arrhythmia detection and classification in ambulatory electrocardiograms using a deep neural network,” Nat Med. 25, 65-69 (2019), a cardiologist-level arrhythmia detection system was developed for diagnosing twelve different types of cardiac rhythms based on a single-lead ECG dataset.

However, the studies in the abovementioned articles are both based on single-lead ECG records, which provide limited information that limits diagnostic accuracy.

### SUMMARY

Therefore, an object of the disclosure is to provide a method that can alleviate at least one of the drawbacks of the prior art.

According to the disclosure, the method for building a heart rhythm classification model that is used to classify a heart rhythm is provided. The method is implemented by a computer device, and includes steps of: A) providing a neural network model that includes first to M^{th} bidirectional long short-term memory (LSTM) layers, M being a positive integer greater than one, wherein each of the first to M^{th} bidirectional LSTM layers includes a first set of LSTM neurons for forward data input and a second set of LSTM neurons for reverse data input, and wherein, for an m^{th} bidirectional LSTM layer where m is an arbitrary one of integers from two to M, each of the LSTM neurons is connected to all of the LSTM neurons of an (m−1)^{th} bidirectional LSTM layer for receiving outputs thereof; B) receiving a plurality of 12-lead electrocardiogram (ECG) datasets, wherein each of the 12-lead ECG datasets is acquired by performing 12-lead electrocardiography on a respective person, corresponds to one of a plurality of predetermined class labels that respectively correspond to a plurality of predetermined heart conditions, and includes first to N^{th} data points that are ordered according to a time sequence the first to N^{th} data points are obtained, wherein N is a positive integer; C) for each of the 12-lead ECG datasets, using the neural network model to generate a classification result that indicates, for each of the predetermined heart conditions, whether the 12-lead ECG dataset corresponds to the predetermined heart condition, wherein step C) includes: feeding the 12-lead ECG dataset into the first set of LSTM neurons of the first bidirectional LSTM layer in a forward sequence from the first data point to the N^{th} data point of the 12-lead ECG dataset, and feeding the 12-lead ECG dataset into the second set of LSTM neurons of the first bidirectional LSTM layer in a reverse sequence from the N^{t}n data point to the first data point of the 12-lead ECG dataset; D) acquiring classification accuracies respectively for the predetermined heart conditions based on the classification results respectively obtained for the 12-lead ECG datasets and the predetermined class labels; and E) when any one of the classification accuracies acquired in step D) is lower than a respective predetermined first threshold, adjusting parameters of the neural network model, and repeating steps C) and D) using the neural network model thus adjusted; and F) making the neural network model serve as the heart rhythm classification model when each of the classification accuracies acquired in step D) is equal to or higher than the respective predetermined first threshold.

### BRIEF DESCRIPTION OF THE DRAWINGS

Other features and advantages of the disclosure will become apparent in the following detailed description of the embodiment(s) with reference to the accompanying drawings, of which:

FIG. 1 is a block diagram illustrating a neural network model used in an embodiment of a method for building a heart rhythm classification model according to this disclosure;

FIG. 2 is a block diagram illustrating a long short-term memory neuron;

FIG. 3 is a flow chart illustrating steps of the embodiment;

FIG. 4 is a plot illustrating an exemplary 12-lead ECG; and

FIGS. 5 and 6 are schematic diagrams that show exemplary confusion matrices for multiple predetermined heart conditions.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

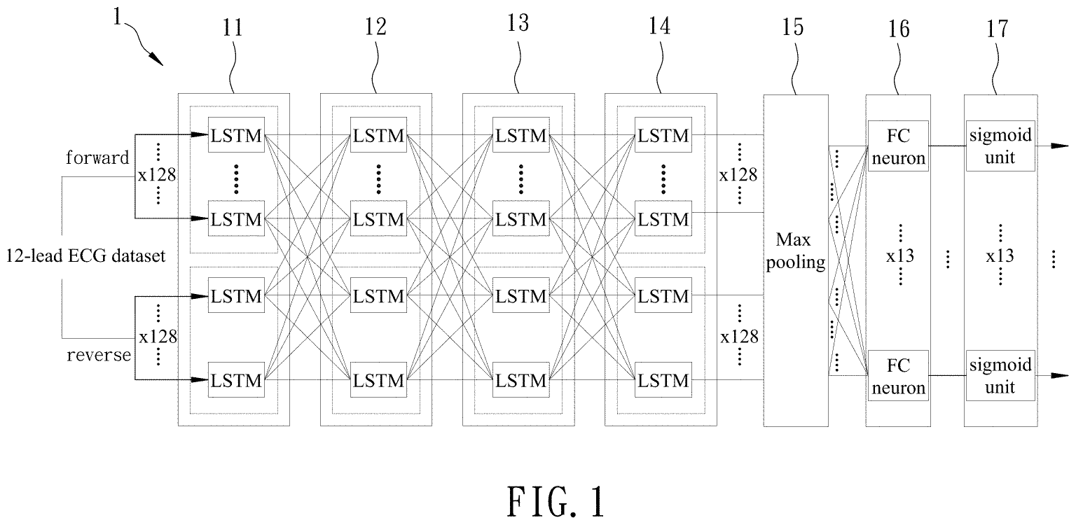

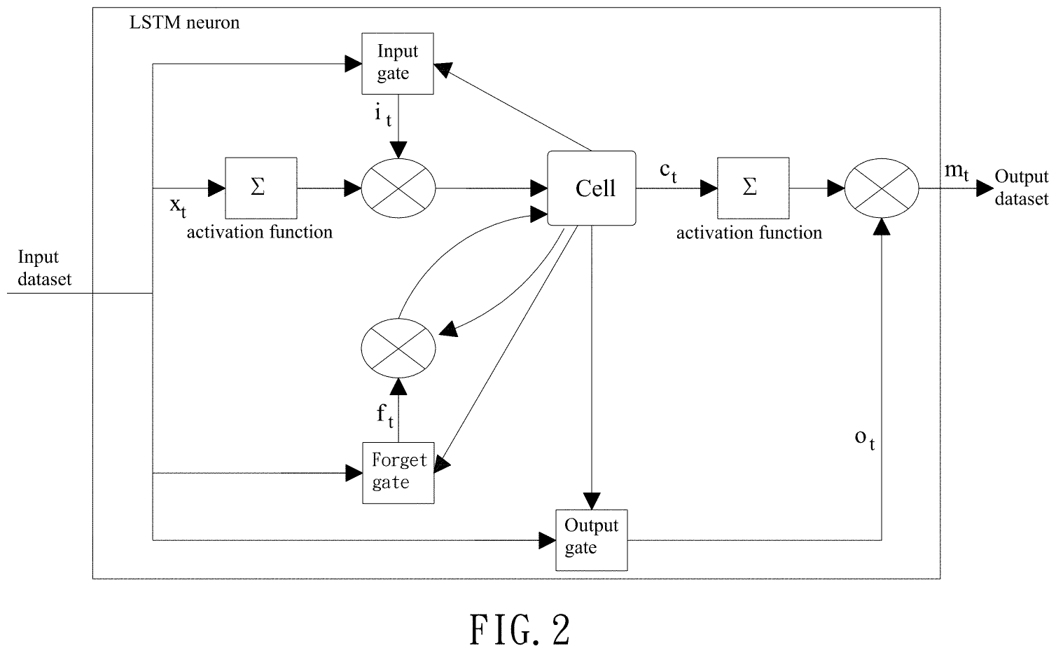

FIG. 1 shows an exemplary neural network model 1 that is used in an embodiment of a method for building a heart rhythm classification model according to this disclosure. The heart rhythm classification model is built by training the neural network model 1, and is used to classify a heart rhythm measured from a person into at least one of a plurality of predetermined heart conditions. In this embodiment, the predetermined heart conditions include thirteen heart conditions in total, which are atrial fibrillation (AFIB), atrial flutter (AFL), atrial premature beat (APB), ventricular bigeminy (BIGEMINY), complete AV (short for atrioventricular) block (or complete heart block, CHB), ectopic atrial rhythm (EAR), first-degree AV block (FRAV), normal sinus rhythm (NSR), paroxysmal supraventricular tachycardia (PSVT), second-degree AV block (SAV), sinus tachycardia (ST), ventricular premature beat (VPB), and ST elevation myocardial infarction (STEMI), but this disclosure is not limited in this respect. The neural network model 1 includes M number of bidirectional long short-term memory (LSTM) layers (referred to as first to M^{th} LSTM layers hereinafter) that are connected one by one, a max pooling layer 15 that is connected to the last one of the bidirectional LSTM layer (i.e., the M^{th} bidirectional LSTM layer in this embodiment), a fully connected (FC) layer 16 that is connected to the max pooling layer 15, and a sigmoid operation block 17 that is connected to the fully connected layer 16, where M is a positive integer greater than one. In this embodiment, M is exemplified to be four, i.e., the neural network model 1 is exemplified to include first to fourth bidirectional LSTM layers 11, 12, 13, 14. Each of the first to M^{th} bidirectional LSTM layers includes a first set of LSTM neurons for forward data input, and a second set of LSTM neurons for reverse data input. In this embodiment, each of the first and second sets of LSTM neurons is exemplified to include 128 LSTM neurons, so each of the bidirectional LSTM layers 11-14 includes 256 LSTM neurons in total. For each of the bidirectional LSTM layers, each of the LSTM neurons thereof receives one or more input datasets each including a plurality of input data points (e.g., first to N^{th} input data points, where N is a positive integer greater than one) that are ordered in a specific time sequence, and outputs an output dataset including a plurality of output data points that are outputted and ordered in sequence, where each of the output data points is a value that is calculated by the LSTM neuron based on the input dataset(s). FIG. 2 is a block diagram illustrating a structure of a single LSTM neuron, which is well known in the art, and details thereof are omitted herein for the sake of brevity.

For the first bidirectional LSTM layer, the inputted 12-lead ECG dataset serves as the input dataset for the LSTM neurons thereof. The 12-lead ECG dataset includes a plurality of ECG data points (i.e., input data points of the input dataset for the first bidirectional LSTM layer) that are ordered according to a time sequence the ECG data points are measured. For an m^{th} bidirectional LSTM layer where m is an arbitrary one of integers from two to M (including two and M), each of the LSTM neurons is connected to all of the LSTM neurons of an (m−1)^{th} bidirectional LSTM layer for receiving the output datasets thereof. Accordingly, the output datasets of the LSTM neurons of the (m−1)^{th} bidirectional LSTM layer serve as the input datasets for each of the LSTM neurons of the m^{th} bidirectional LSTM layer.

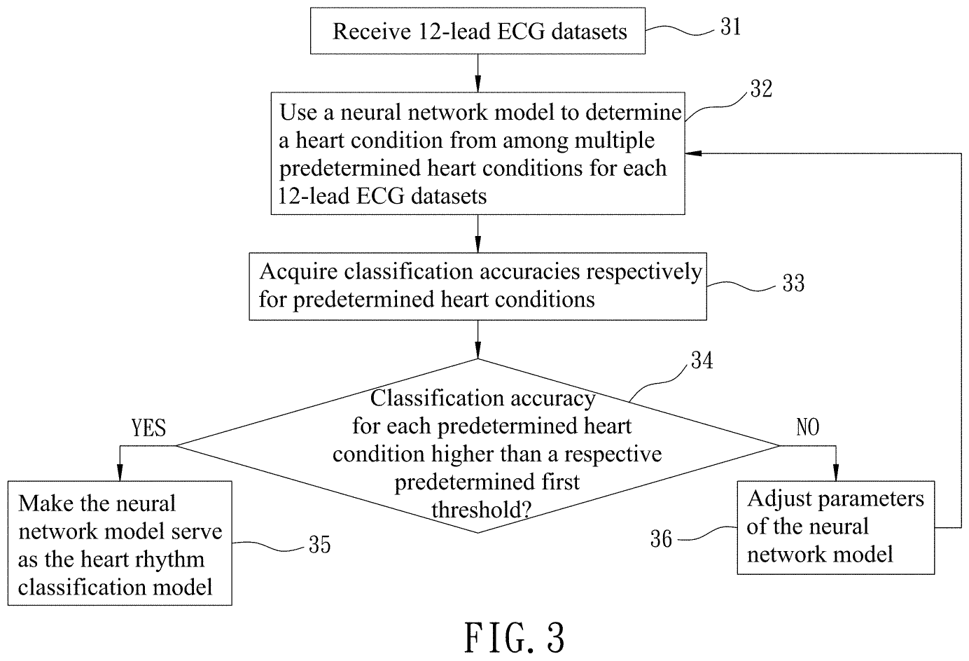

The embodiment of the method for building the heart rhythm classification model is implemented by a computer device that stores the neural network model or that is communicatively connected to a system (e.g., a cloud system) storing the neural network model which is to be executed by the computer device. Referring to FIG. 3, the embodiment of the method for building the heart rhythm classification model according to this disclosure includes steps 31-36.

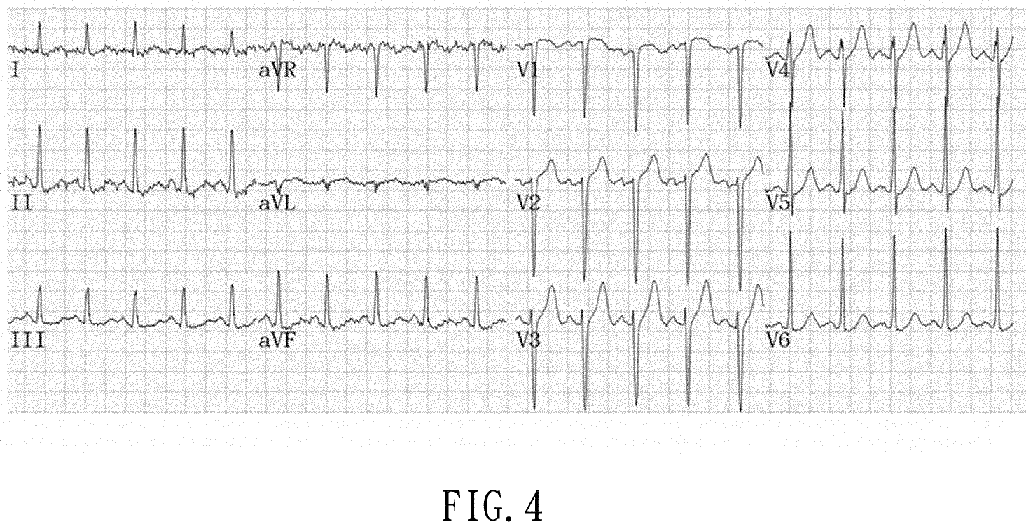

In step 31, the computer device receives a plurality of 12-lead electrocardiogram (ECG) datasets. Each of the 12-lead ECG datasets is acquired by performing 12-lead electrocardiography on a respective person for a predetermined period of time, such as 10 seconds, and is provided with a predetermined class label that corresponds to at least one of the predetermined heart conditions. For each of the 12-lead ECG datasets, the predetermined class label is given via a consensus of multiple board-certified electrophysiologists, and serves as a gold standard for verifying a classification result of the neural network model 1. Each ECG data point may be a vector that indicates magnitudes of the heart's electrical potential measured respectively from twelve leads at a corresponding time point, where the twelve leads include the three standard limb leads (I, II and III), three augmented limb leads (aVR, aVL and aVF) and six precordial leads (V1, V2, V3, V4, V5 and V6). FIG. 4 shows an exemplary 12-lead ECG that corresponds to a time period of 2.5 seconds.

Referring to FIGS. 1 and 3 again, in step 32, the computer device uses the neural network model 1 to generate, for each of the 12-lead ECG datasets, a classification result that indicates, for each of the predetermined heart conditions, whether the 12-lead ECG dataset corresponds to the predetermined heart condition. The following descriptions explain the way of using the neural network model 1 to classify a single 12-lead ECG dataset.

In each of the bidirectional LSTM layers, bidirectional data processing is performed, which means that the computer device makes the LSTM neurons in the first set receive the corresponding input dataset(s) in a forward sequence that is the same as the sequence the input data points of each of the input dataset(s) are ordered (i.e., from the first input data point to the last input data point in a single input dataset), and makes the LSTM neurons in the second set receive the corresponding input dataset(s) in a reverse sequence that is opposite to the sequence the input data points of each of the input dataset(s) are ordered (i.e., from the last input data point to the first input data point in a single input dataset). Referring to FIG. 1, as an example, for each of the 12-lead ECG datasets, the computer device feeds the 12-lead ECG dataset into the first set (upper part) of LSTM neurons of the first bidirectional LSTM layer 11 in the forward sequence (i.e., from the first data point to the N^{th} data point of the 12-lead ECG dataset), and feeds the 12-lead ECG dataset into the second set (lower part) of LSTM neurons of the first bidirectional LSTM layer 11 in a reverse sequence (i.e., from the N^{th} data point to the first data point of the 12-lead ECG dataset). By virtue of the bidirectional data processing, the neural network model 1 may extract more features for classification of the 12-lead ECG dataset, and the resultant heart rhythm classification model, which is the trained neural network model, may have enhanced accuracy in classification.

In the max pooling layer 15, the computer device acquires a feature value for each of the LSTM neurons in the first set and the second set of the M^{th} bidirectional LSTM layer. In this embodiment, the computer device performs max pooling to acquire, for each of the LSTM neurons of the M^{th} bidirectional LSTM layer, the greatest one of the output data points of the output dataset of the LSTM neuron to be the feature value.

The fully connected layer 16 receives the feature values from the max pooling layer 15, and includes a plurality of fully connected neurons that respectively correspond to the predetermined heart conditions. Therefore, the fully connected layer 16 includes thirteen fully connected neurons that respectively correspond to the thirteen predetermined heart conditions in this embodiment. Each of the fully connected neurons includes a plurality of weights. In each of the fully connected neurons of the fully connected layer 16, the computer device performs inner product operation on the feature values and the weights of the fully connected neuron to obtain a fully connected feature value that is related to the corresponding one of the predetermined heart conditions. It is noted that, in each of the fully connected neurons, the computer device may further perform linear operation on the result of the inner product operation to obtain the fully connected feature value, but this disclosure is not limited in this respect.

The sigmoid operation block 17 includes multiple sigmoid units that correspond to the predetermined heart conditions. Each of the sigmoid units is connected to a respective one of the fully connected neurons for receiving the fully connected feature value generated thereby. In each of the sigmoid units, the computer device applies a sigmoid function to the fully connected feature value that is obtained for the corresponding one of the fully connected neurons to obtain an index value that ranges between 0 and 1 for the corresponding one of the predetermined heart conditions. The sigmoid function is generally expressed by:

$$σ ( {z}_{{J}^{´}} ) = \frac{{e}^{{z}_{j}}}{1 + {e}^{{z}_{j}}} , for j = 1 , … , K$$

where K is a number of inputs. In this case, each of the sigmoid units is connected to only one of the fully connected neurons, so K=1, and the sigmoid function can be modified to be:

$$σ ( x ) = \frac{{e}^{x}}{1 + {e}^{x}}$$

where x is the fully connected feature value generated by the corresponding one of the fully connected neurons.

Then, the computer device generates, based on the index values obtained for the predetermined heart conditions, the classification result that indicates, for each of the predetermined heart conditions, whether the 12-lead ECG dataset corresponds to the predetermined heart condition. In practice, a plurality of comparison thresholds may be provided for the predetermined heart conditions, respectively. For each of the predetermined heart conditions, the computer device compares the corresponding one of the index values with the corresponding one of the comparison thresholds, determines that the 12-lead ECG dataset corresponds to the predetermined heart condition when the corresponding one of the index values is greater than the corresponding one of the comparison thresholds, and determines that the 12-lead ECG dataset does not correspond to the predetermined heart condition when otherwise. In general cases, the comparison thresholds may be set to 0.5. In some cases, each of the comparison thresholds may be adjusted as desired, so the comparison thresholds may be different for different predetermined heart conditions.

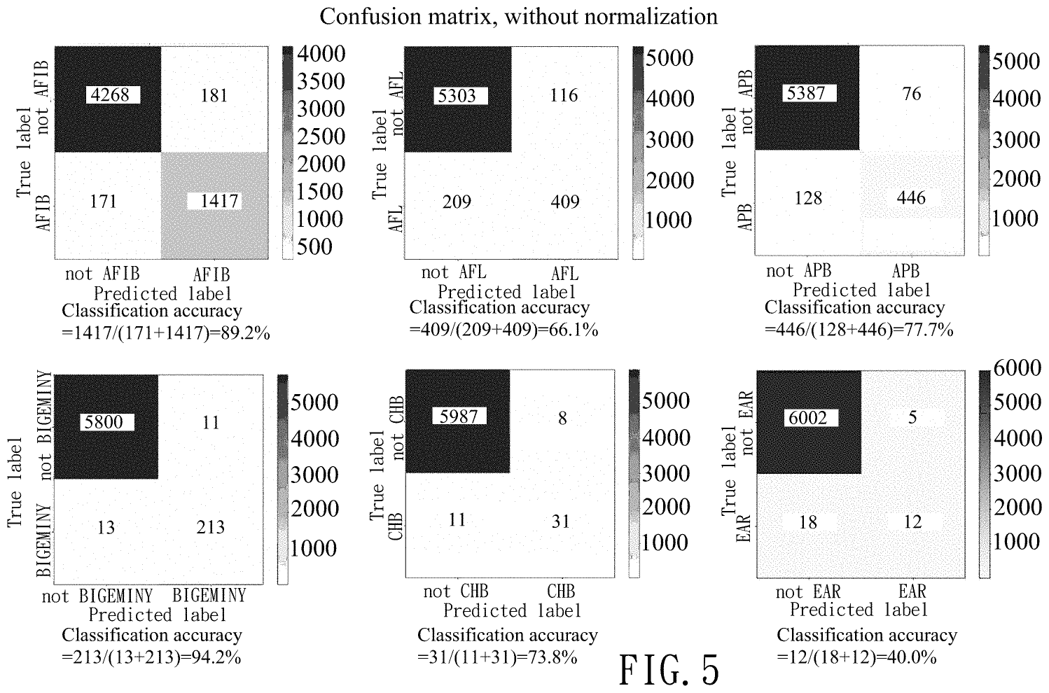

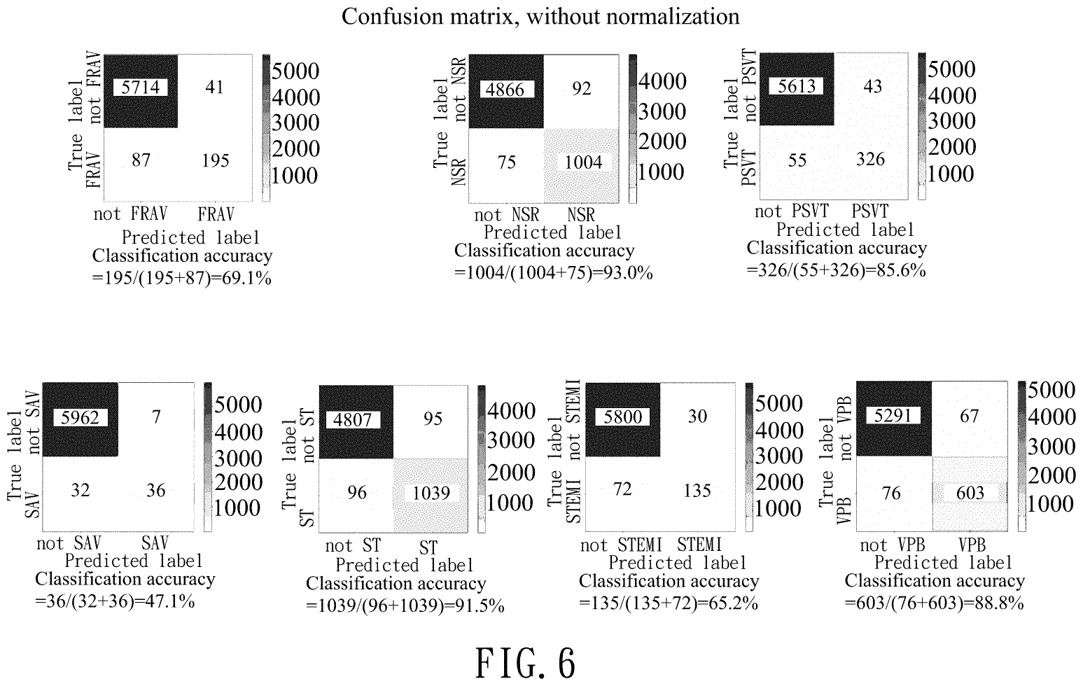

After generating the classification result for each of the 12-lead ECG datasets, in step 33, the computer device acquires classification accuracies respectively for the predetermined heart conditions based on the classification results respectively obtained for the 12-lead ECG datasets and the predetermined class labels. In detail, the computer device determines, for each of the predetermined heart conditions, whether the classification result generated for each of the 12-lead ECG datasets accurately indicates a correspondence between the 12-lead ECG dataset and the predetermined heart condition by comparing the classification result and the predetermined class label provided for the 12-lead ECG dataset, so as to determine the classification accuracies respectively for the predetermined heart conditions. FIGS. 5 and 6 exemplarily show confusion matrices for the thirteen predetermined heart conditions, respectively. The numbers in each confusion matrix show the sample quantities that are accurately or inaccurately classified, so the classification accuracy (referred to as the true positive rate in this embodiment, but this disclosure is not limited in this respect) for the predetermined heart condition can be calculated accordingly.

In step 34, the computer device determines, for each of the predetermined heart conditions, whether the corresponding one of the classification accuracies is higher than a respective predetermined first threshold. Since the difficulties for identifying different heart conditions may vary, the predetermined heart conditions may correspond to different predetermined first thresholds, which can be defined by the model developer as desired. The flow goes to step 35 when every single one of the classification accuracies is higher than or equal to the respective predetermined first threshold, and goes to step 36 when any one of the classification accuracies is lower than the respective predetermined first threshold.

In step 35, the computer device makes the neural network model serve as the heart rhythm classification model, which can be used to make diagnosis on a heart condition of a person by inputting a 12-lead ECG dataset measured from the person into the heart rhythm classification model.

In step 36, the computer device adjusts parameters (i.e., weights of each neuron) of the neural network model 1, and the flow goes back to step 32 to repeat operations of steps 32 and 33 using the neural network model 1 thus adjusted. Adjustment can be made to parameters/weights of the bidirectional LSTM layers and/or the fully connected layer 16 based on conventional algorithms such as gradient descents, or on the experience of the model developer. In one embodiment, the computer device adjusts the weights of the fully connected neurons based on the classification accuracies. In detail, for one of the predetermined heart conditions of which the corresponding classification accuracy is lower than a predetermined second threshold, the adjustment made to the weights of one of the fully connected neurons that corresponds to said one of the predetermined heart conditions is greater than the adjustment made to the weights of one of the fully connected neurons that corresponds to one of the predetermined heart conditions of which the corresponding classification accuracy is equal to or higher than the predetermined second threshold. Taking the confusion matrices shown in FIGS. 5 and 6 as an example, if the predetermined first threshold and predetermined second threshold are set respectively to 70% and 65%, the fully connected neurons that correspond to EAR and SAV, of which the corresponding classification accuracies are 40.0% and 47.1%, respectively, would have larger adjustments in weights in comparison to other fully connected neurons. The predetermined second threshold may be defined by the model developer as desired.

In summary, the embodiment of the method for building a heart rhythm classification model according to this disclosure uses 12-lead ECG datasets to classify heart rhythms. 12-lead ECG datasets contain more information than single-lead ECG datasets, so the classification accuracies can be enhanced. The embodiment further uses the bidirectional LSTM layers to make the neural network model or the heart rhythm classification model analyze 12-lead ECG datasets from two different aspects, so the model may obtain more features from the 12-lead ECG datasets, thereby further enhancing the classification accuracies for the predetermined heart conditions.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for building a heart rhythm classification model that is used to classify a heart rhythm, the method implemented by a computer device and comprising steps of:
A) providing a neural network model that includes first to M^{th} bidirectional long short-term memory (LSTM) layers, M being a positive integer greater than one,
wherein each of the first to M^{th} bidirectional LSTM layers includes a first set of LSTM neurons for forward data input and a second set of LSTM neurons for reverse data input, and
wherein, for an m^{th} bidirectional LSTM layer where m is an arbitrary one of integers from two to M, each of the LSTM neurons is connected to all of the LSTM neurons of an (m−1)^{th} bidirectional LSTM layer for receiving outputs thereof;
B) receiving a plurality of 12-lead electrocardiogram (ECG) datasets, wherein each of the 12-lead ECG datasets is acquired by performing 12-lead electrocardiography on a respective person, corresponds to one of a plurality of predetermined class labels that respectively correspond to a plurality of predetermined heart conditions, and includes first to N^{th} data points that are ordered according to a time sequence the first to N^{th} data points are obtained, wherein N is a positive integer greater than one;
C) for each of the 12-lead ECG datasets, using the neural network model to generate a classification result that indicates, for each of the predetermined heart conditions, whether the 12-lead ECG dataset corresponds to the predetermined heart condition, wherein step C) includes: feeding the 12-lead ECG dataset into the first set of LSTM neurons of the first bidirectional LSTM layer in a forward sequence from the first data point to the N^{th} data point of the 12-lead ECG dataset, and feeding the 12-lead ECG dataset into the second set of LSTM neurons of the first bidirectional LSTM layer in a reverse sequence from the N^{th} data point to the first data point of the 12-lead ECG dataset;
D) acquiring classification accuracies respectively for the predetermined heart conditions based on the classification results respectively obtained for the 12-lead ECG datasets and the predetermined class labels; and
E) when any one of the classification accuracies acquired in step D) is lower than a respective predetermined first threshold, adjusting parameters of the neural network model, and repeating steps C) and D) using the neural network model thus adjusted; and
F) making the neural network model serve as the heart rhythm classification model when each of the classification accuracies acquired in step D) is equal to or higher than the respective predetermined first threshold.

2. The method of claim 1, wherein the neural network model provided in step A) further includes a max pooling layer that is connected to the M^{th} bidirectional LSTM layers; and
wherein step C) further includes: using the max pooling layer to acquire, for the 12-lead ECG dataset, a feature value for each of the LSTM neurons in the first set and the second set of the M^{th} bidirectional LSTM layer, and the classification result that corresponds to the 12-lead ECG dataset is determined based on the feature values acquired for the LSTM neurons in the first set and the second set of the M^{th} bidirectional LSTM layer.

3. The method of claim 2, wherein the neural network model provided in step A) further includes a fully connected layer that is connected to the max pooling layer for receiving the feature values therefrom, and that includes a plurality fully connected neurons respectively corresponding to the predetermined heart conditions;
wherein each of the fully connected neurons includes a plurality of weights, and step C) further includes: for the 12-lead ECG dataset, using each of the fully connected neurons to perform inner product operation on the feature values and the weights of the fully connected neuron to obtain a fully connected feature value that is related to a probability of the corresponding one of the predetermined heart conditions; and
wherein, in step C), the classification result is determined based on the fully connected feature values obtained for the LSTM neurons in the first set and the second set of the M^{th} bidirectional LSTM layer.

4. The method of claim 3, wherein step C) further includes: for the 12-lead ECG dataset, applying a sigmoid function to each of the fully connected feature values obtained for the fully connected neurons to obtain a plurality of index values respectively for the predetermined heart conditions, and determining the classification result based on the index values.

5. The method of claim 4, the predetermined heart conditions respectively corresponding to a plurality of comparison thresholds, wherein step C) further includes, for each of the predetermined heart conditions:
comparing the corresponding one of the index values with the corresponding one of the comparison thresholds,
determining that the 12-lead ECG dataset corresponds to the predetermined heart condition when the corresponding one of the index values is greater than the corresponding one of the comparison thresholds, and
determining that the 12-lead ECG dataset does not correspond to the predetermined heart condition when the corresponding one of the index values is not greater than the corresponding one of the comparison thresholds.

6. The method of claim 3, wherein, in step E), the adjusting includes:
adjusting the weights of the fully connected neurons based on the classification accuracies, wherein, for one of the predetermined heart conditions of which the corresponding one of the classification accuracies is lower than a predetermined second threshold, the adjustment made to the weights of one of the fully connected neurons that corresponds to the predetermined heart condition is greater than the adjustment made to the weights of one of the fully connected neurons that corresponds to one of the predetermined heart conditions of which the corresponding one of the classification accuracies is equal to or higher than the predetermined second threshold.

7. The method of claim 1, wherein step D) includes: determining, for each of the predetermined heart conditions, whether the classification result generated for each of the 12-lead ECG datasets accurately indicates a correspondence between the 12-lead ECG dataset and the predetermined heart condition by comparing the classification result and the predetermined class label provided for the 12-lead ECG dataset, so as to determine the classification accuracies respectively for the predetermined heart conditions.
